(19) United States
(12) Patent Application Publication
(10) Pub. No.: US 2024/0398541 A1
(43) Pub. Date: Dec. 5, 2024

(54) SPLANCHNIC FLOW REGULATION IMPLANTS

(71) Applicant: EDWARDS LIFESCIENCES CORPORATION, Irvine, CA (US)

(72) Inventors: Arnold Cruz Tuason, Claremont, CA (US); Emil Karapetian, Huntington Beach, CA (US); Sam Chitsaz, Walnut Creek, CA (US)

(21) Appl. No.: 18/653,699

(22) Filed: May 2, 2024

Related U.S. Application Data
(63) Continuation of application No. PCT/US2022/048533, filed on Nov. 1, 2022.
(60) Provisional application No. 63/276,505, filed on Nov. 5, 2021.

(51) Int. Cl. A61F 2/07; A61F 2/00; A61F 2/06

(52) U.S. Cl. CPC A61F 2/07; A61F 2002/0081; A61F 2002/065; A61F 2002/068; A61F 2230/0078; A61F 2250/001

(57) ABSTRACT

A medical implant for managing blood flow comprises a frame configured for deployment within a first blood vessel and configured to extend at least partially across a first in-flow junction of a second blood vessel, the frame at least partially composed of wire-like struts configured to allow at least partial blood flow through gaps between struts.

### RELATED APPLICATION

This application is a continuation of International Patent Application No. PCT/US2022/048533, filed Nov. 1, 2022, and entitled SPLANCHNIC FLOW REGULATION IMPLANTS, which claims priority to U.S. Provisional Application No. 63/276,505, filed Nov. 5, 2021, and entitled SPLANCHNIC FLOW REGULATION IMPLANTS, the disclosure of which is hereby incorporated by reference in its entirety.

### BACKGROUND

The present invention relates generally to the field of medical devices and procedures.

Redistribution of blood from the splanchnic venous circulation to the inferior vena cava (IVC) can contribute to increases in central venous pressure (CVP), pulmonary artery pressure, and/or pulmonary capillary wedge pressure (PCWP), particularly during periods of elevated sympathetic tone (e.g., exercise) in heart failure patients.

### SUMMARY

Described herein are devices, methods, and systems that facilitate blood flow management through and/or into one or more blood vessels of a heart.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 provides a schematic representation of portions of the splanchnic circulation.

FIG. 2 provides another schematic representation of the splanchnic circulation, illustrating blood flow from the aorta to the inferior vena cava (IVC).

FIG. 3 illustrates portions of the splanchnic venous circulation acting as a blood reservoir between the aorta and the IVC.

FIG. 4 illustrates blood flow from one or more splanchnic arteries through the splanchnic reservoir, into a hepatic vein and finally into the IVC.

FIG. 5 illustrates an example flow-regulating implant configured for placement at least partially within an IVC of a patient in accordance with one or more examples.

FIG. 6 illustrates an example flow-regulating implant configured for placement at least partially within an IVC of a patient in accordance with one or more examples.

FIG. 7 illustrates an example flow-regulating implant deployed at least partially within an IVC in accordance with one or more examples.

FIG. 8 illustrates an example flow-regulating implant deployed at least partially within an IVC in accordance with one or more examples.

FIGS. 9A and 9B illustrate an example flow-regulating implant configured to placement at least partially within a hepatic vein to limit blood flow into an IVC in accordance with one or more examples.

FIGS. 10-1, 10-2, and 10-3 are flowcharts including steps of a process for delivering one or more implants in accordance with one or more examples.

FIGS. 11-1, 11-2, and 11-3 provide images relating to various steps of the process of FIG. 10.

FIG. 12 illustrates another example flow-regulating implant configured to placement at least partially within a hepatic vein to limit blood flow into an IVC in accordance with one or more examples.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

### Overview

The following includes a general description of human cardiac anatomy that is relevant to certain inventive features and examples disclosed herein and is included to provide context for certain aspects of the present disclosure.

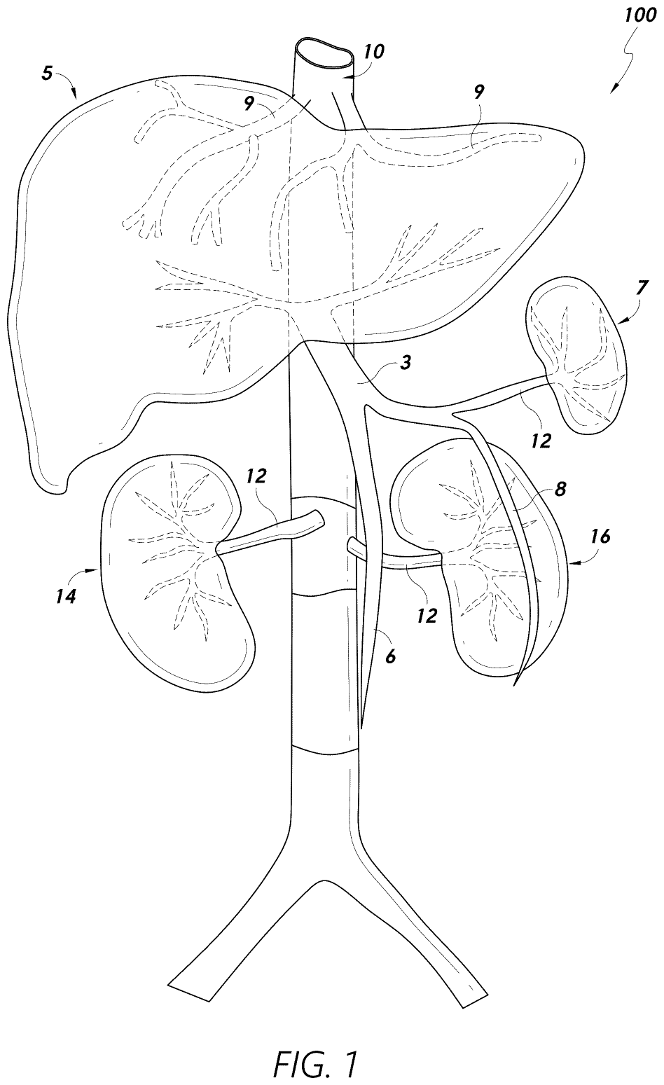

FIG. 1 provides a schematic representation of portions of the splanchnic circulation 100. The term “splanchnic circulation” refers to blood flow originating from the celiac, superior mesenteric, and inferior mesenteric arteries to the abdominal gastrointestinal organs. The splanchnic circulation 100 receives approximately 25% of the cardiac output and holds a similar percentage of the total blood volume under normal conditions. The splanchnic circulation 100 can act as a site of cardiac output regulation and/or as a blood reservoir. Multiple regulatory pathways are involved in the distribution of the splanchnic circulation.

Total flow to the splanchnic viscera is controlled by resistance vessels in the mesenteric and hepatic arterial systems. The venous effluents from the splanchnic viscera converge to form the portal vein 3, which supplies approximately 75% of the total blood supply to the liver 5. The portal blood not only is high in substrate concentrations resulting from intestinal absorption but also tends to contain bacteria and endotoxin.

Renal veins 12 drain blood from the right kidney 14 and left kidney 16 and connect to the inferior vena cava 10 (IVC). The superior mesenteric vein 6 is a major venous tributary of the abdominal cavity that lies laterally to the superior mesenteric artery and serves to drain the vast majority of the organs of the abdominal cavity. The inferior mesenteric vein 8 drains blood from the large intestine. The splenic vein 12 is a blood vessel that drains blood from the spleen, the stomach fundus, and part of the pancreas.

The portal vein 3 receives blood from the stomach, intestines, pancreas, and spleen 7 and carries it into the liver 5 through the porta hepatis. The porta hepatis serves as the point of entry for the portal vein 3 and the proper hepatic artery, and is the point of exit for the bile passages.

Following processing of the blood by the liver 5, the blood collects in the central vein at the core of the lobule. Blood from these central veins ultimately converges in the right and left hepatic veins 9, which exit the superior surface of the liver 5 and empty into the IVC 10 to be distributed to the rest of the body.

The splanchnic venous circulation 100 is highly compliant and can act as a blood reservoir that can be recruited in order to support the need for increased stressed blood volume during periods of elevated sympathetic tone, such as exertion, in order to support increased cardiac output and vasodilation of peripheral vessels supporting active muscles. However, heart failure patients can have multiple comorbidities that prevent them from using that additional blood volume. Such comorbidities can include chronotropic incompetence, inability to increase stroke volume, and/or peripheral microvascular dysfunction. This can lead to venous congestion and/or abrupt rises in pulmonary capillary wedge pressure (PCWP).

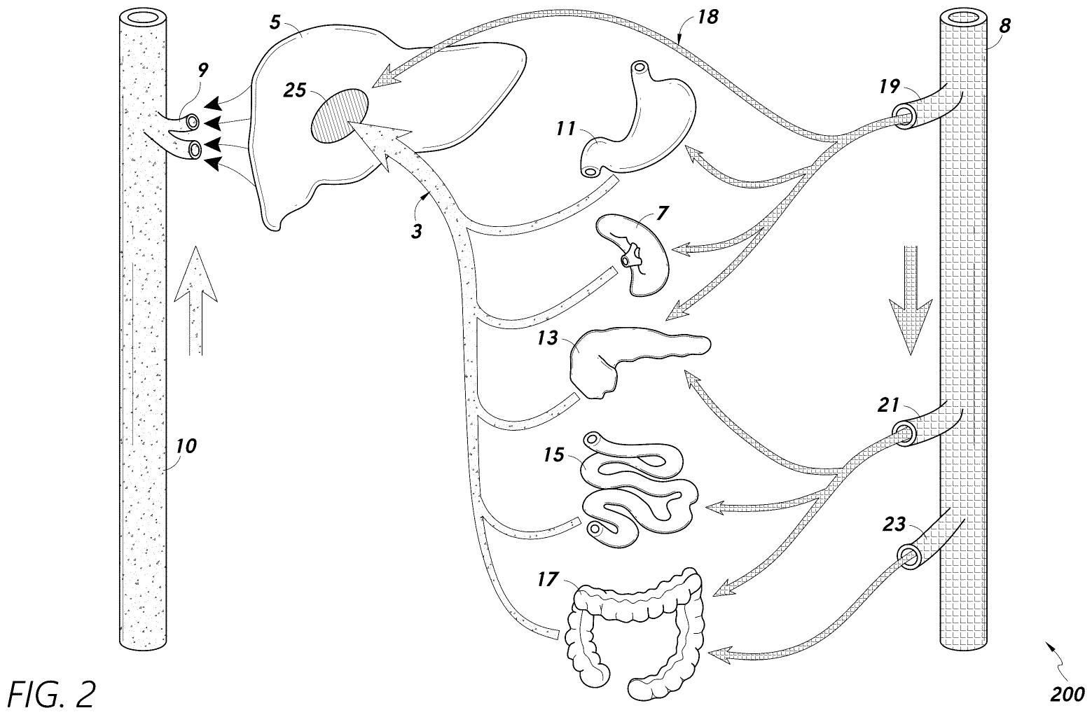

FIG. 2 provides another schematic representation of the splanchnic circulation 200, illustrating blood flow from the aorta 8 to the IVC 10. Blood travels from the aorta 8 to the abdominal gastrointestinal organs including the stomach 11, liver 5, spleen, 7, pancreas 13, small intestine 15, and large intestine 17. The splanchnic circulation 200 comprises three major branches of the abdominal aorta 9, including the coeliac artery 19, the superior mesenteric artery 21 (SMA), and the inferior mesenteric artery 23 (IMA). The hepatic portal circulation (e.g., the hepatic artery 18 and/or portal vein 3) delivers the majority of blood flow to the liver 5.

The coeliac artery 19 is the first major division of the abdominal aorta 8, branching at T12 in a horizontal direction ˜1.25 cm in length. It shows three main divisions such as the left gastric artery, common hepatic artery 18, and splenic artery and is the primary blood supply to the stomach 11, upper duodenum, spleen 7, and pancreas 13.

The SMA 21 arises from the abdominal aorta 8 anteriorly at L1, usually 1 cm inferior to the coeliac artery 19. The five major divisions of the SMA 21 are the inferior pancreaticoduodenal artery, intestinal arteries, ileocolic, right colic, and middle colic arteries. The SMA 21 supplies the lower part of the duodenum, jejunum, ileum, caecum, appendix, ascending colon, and two-thirds of the transverse colon. It is the largest of the splanchnic arterial vessels delivering >10% of the cardiac output and therefore has significant implications for embolic mesenteric ischaemia.

The IMA 23 branches anteriorly from the abdominal aorta 8 at L3, midway between the renal arteries and the iliac bifurcation. The main branches of the IMA 23 are the left colic artery, the sigmoid branches, and the superior rectal artery. It forms a watershed with the middle colic artery and supplies blood to the final third of the transverse colon, descending colon, and upper rectum.

Blood flow is conveyed into the liver 5 via the portal vein 3 into sinusoids 25 of the liver 5. The hepatic veins 9 convey the blood from the liver 5 to the IVC 10.

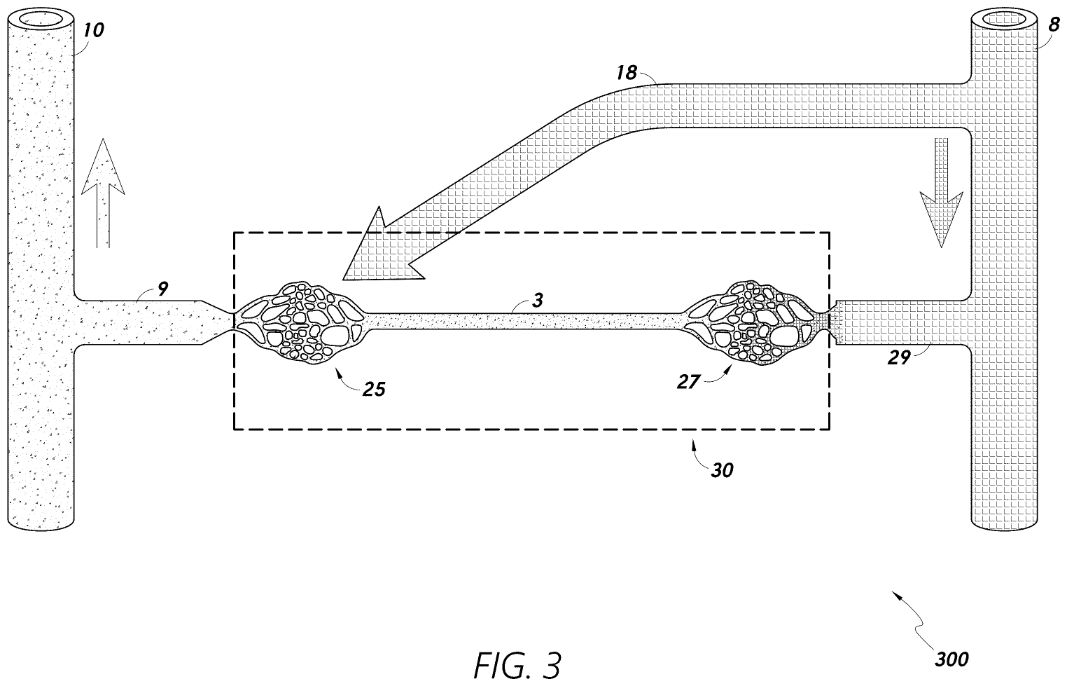

FIG. 3 illustrates portions of the splanchnic venous circulation 300 acting as a blood reservoir 30 between the aorta 8 and the IVC 10. The portal vein 30 conveys blood between the splanchnic organs 27 (e.g., the stomach, spleen, etc.) and the liver sinusoids 25. The liver sinusoids 25 also receive blood from the hepatic artery 18. The splanchnic organs 27 receive blood from the aorta 8 via various splanchnic arteries 29 (e.g., the SMA, IMA, etc.). The amount of blood contained in the portal vein 3 at any given time can be variable.

For some patients (especially patients experiencing heart failure) fluid redistribution from the splanchnic venous reservoir 30 to the IVC 10 and/or stressed blood volume can contribute to increases in central venous pressure (CVP), pulmonary artery pressure, and/or PCWP. This can be especially problematic during periods of elevated sympathetic tone, such as exertion, and/or can lead to pulmonary congestion that can impact a patient's quality of life and/or can lead to acute decompensation.

The splanchnic venous circulation 300, and particularly the portal vein 3, can advantageously provide a blood reserve to support the need for increased stressed blood volume during periods of elevated sympathetic tone. Because blood flow from the splanchnic venous circulation 300 is directed through the hepatic veins 9 and into the IVC 10, devices placed into the hepatic veins 9 and/or IVC 10 to limit blood flow can allow the reservoir 30 to expand with increased blood volume.

Examples described herein can relate to devices and/or methods that can advantageously limit, stagnate, and/or impede blood flow into the IVC 10 from the hepatic veins 9 to increase the pressure gradient between the IVC 10 and the liver and/or splanchnic venous circulation 300. In some examples, one or more flow-regulating implants may be configured for placement at least partially within the hepatic veins 9 and/or IVC 10 and/or at one or more junctions between the hepatic veins 9 and the IVC 10. As a result, blood flowing from the splanchnic venous reservoir 30 into the hepatic veins 9 can be slowed to increase blood volume in the splanchnic venous reservoir 30.

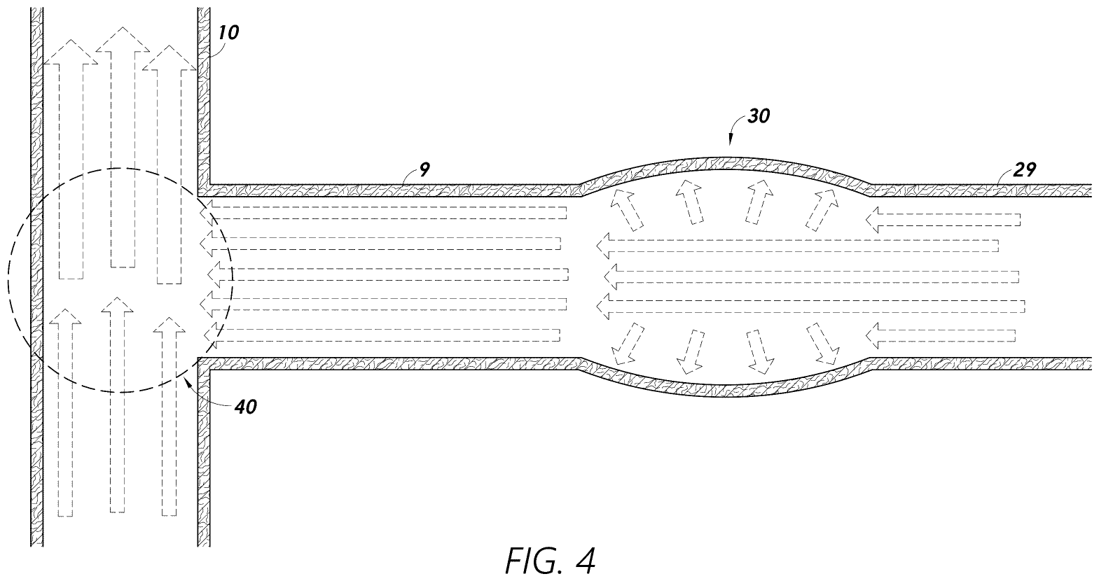

FIG. 4 illustrates blood flow from one or more splanchnic arteries 29 through the splanchnic reservoir 30, into a hepatic vein 9 and finally into the IVC 10. While only a single hepatic vein 9 is shown in FIG. 4 for illustrative purposes, multiple hepatic veins may convey blood from the reservoir into the IVC 10. The hepatic vein 9 may feed into a junction portion 40 of the IVC 10. Accordingly, to limit blood flow into the IVC 10, one or more flow-limiting implants may be configured for placement within the hepatic vein 9 and/or at least partially within the junction portion 40 of the IVC 10.

As shown in FIG. 4, as blood volume within the reservoir 30 increases, blood may press against the walls of the reservoir (e.g., against the walls of the portal vein). The compliance of the reservoir 30 may allow the reservoir 30 to expand in response to the increased blood volume.

The present disclosure provides methods and devices (including various medical implants) for managing blood flow within a human body. The term “implant” is used herein according to its plain and/ordinary meaning and may refer to any medical implant, frame, valve, shunt, stent, anchor, and/or similar devices for use in treating various conditions in a human body. Implants may be delivered percutaneously and/or via catheter (i.e., transcatheter) for various medical procedures and may have a generally sturdy and/or flexible structure. The term “catheter” is used herein according to its broad and/ordinary meaning and may include any tube, sheath, steerable sheath, steerable catheters, and/or any other type of elongate tubular delivery device comprising an inner lumen configured to slidably receive instrumentation, such as for positioning within an IVC and/or hepatic vein, including for example delivery catheters and/or cannulas.

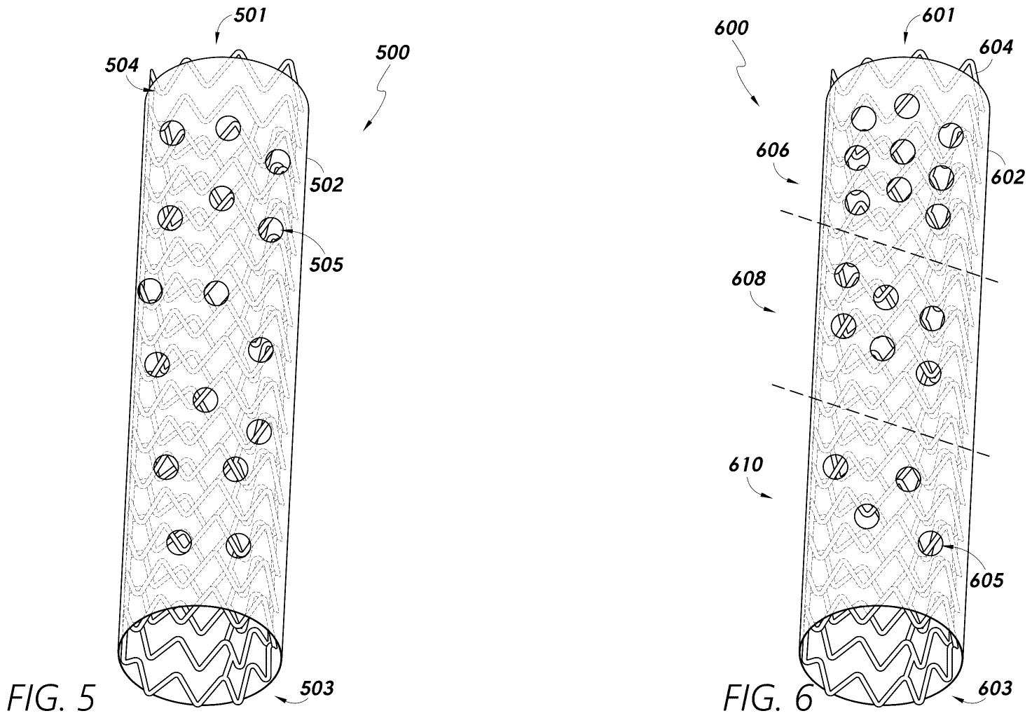

FIG. 5 illustrates an example flow-regulating implant 500 configured for placement at least partially within an IVC of a patient in accordance with one or more examples. The implant 500 may be configured to extend within and/or along at least a portion of an IVC and/or at an in-flow portion of the IVC. For example, the implant 500 may be configured to expand at least partially across one or more junction portions between one or more hepatic veins and the IVC.

The flow-regulating implant 500 may be configured for reducing hepatic vein blood flow into the IVC. The implant can have a tubular and/or cylindrical form to approximate a shape of the IVC and/or can provide radial blood flow regulation. For example, the implant 500 may be configured to regulate blood that is generally perpendicular and/or into the sides of the implant 500. The implant can comprise one or more openings 505 and/or apertures which can allow the implant 500 to partially block blood flow while allowing partial flow through the sides of the implant 500. The number and/or size of the openings 505 can vary along the implant 500 to allow for adjustment of flow rate based on placement of the implant 500.

The implant 500 may be configured to be positioned to cover one or more junctions between the IVC and at least one hepatic vein. In some examples, the implant 500 may be configured to at least partially block and/or allow blood flow from multiple hepatic veins.

In some examples, the implant 500 may comprise a wire frame 504 at least partially enclosed by a covering 502. The frame 504 may comprise a network of one or more interconnected wires and/or wire-like struts forming a tubular and/or cylindrical shape and/or extending around a generally tubular and/or cylindrical lumen. The frame 504 may be composed of one or more materials, which can include one or more shape-memory metallic alloys (e.g., Nitinol). As shown in FIG. 5, the frame 504 and/or segments of the frame 504 may have a generally wavy and/or bent form and/or may be configured to compress and/or expand to facilitate delivery and/or placement of the implant 500 within the IVC. In some examples, the frame 504 may be shape-set in a tubular form and/or may be shaped into the tubular form by the covering 502 and/or otherwise. Segments of the frame 504 may be separated and/or gaps may exist between portions of the frame 504. The frame 504 may comprise a network of wires and/or wire-like struts forming cells and/or gaps between the wires and/or struts. In some examples, the struts may comprise thin and/or elongate metallic wires having a wavy and/or coiled form to facilitate expansion and/or compression of the frame 504.

The covering 502 may be configured to extend over the frame 504 and/or to cover gaps between portions of the frame. In some examples, the covering 502 may have a tubular form and/or may be configured to adhere and/or otherwise attach to the frame 504 to assume the tubular form shown in FIG. 5. The covering 502 may be composed of any suitable material, including various fabrics. In some examples, the covering 502 may be at least partially fluid-tight and/or may be configured to prevent blood flow into the implant 500.

The openings 505 may represent punctures through the covering 502 and/or frame 504. For example, the covering 502 may be configured to be punctured without compromising and/or modifying the form and/or structure of the covering 502. In some examples, the covering 502 may comprise openings 505 at only a portion of the covering 502. For example, only portions of the covering 502 configured for placement over an in-flow tube from a hepatic vein may comprise openings 505. However, the covering 502 may comprise openings 505 at any and/or all portions of the covering 502. While the openings 505 are shown having a circular shape, the openings 505 may have any suitable shape and/or size.

In some examples, the implant 500 may be configured to at least partially obstruct all hepatic vein junctions with the IVC along the length of the implant 500. For example, the implant 500 may be configured to extend across IVC-hepatic vein junctions of two or three hepatic veins and/or may comprise one or more openings 505 at each junction. In some examples, the implant 500 may have a generally flexible and/or bendable structure and/or may otherwise be configured to facilitate delivery into the IVC.

While the covering 502 is shown comprising a single covering 502 with puncture openings through the covering 502, the covering 502 may additionally or alternatively comprise multiple disparate sections. For example, the covering 502 may comprise one or more strips and/or lengths of material with gaps between the strips and/or length of the material to allow blood flow through the gaps. In such examples, the covering 502 and/or coverings 502 may be configured to at least partially block in-flow from one or more hepatic veins and/or to not completely cover in-flow from the one or more hepatic veins. In some examples, the covering 502 may comprise one or more overlapping and/or intersecting strips and/or fibers of material forming gaps between the overlapping and/or intersection strips and/or fibers to allow some measure of blood flow through the covering 502. The covering 502 may comprise one or more cells configured to allow blood flow through the covering 502.

In some examples, the implant 500 may be configured to assume a compressed form while within a delivery device (e.g., a catheter). Following removal from the delivery device, the implant 500 may be configured to at least partially expand and/or otherwise anchor within the IVC. In some examples, the implant 500 may be at least partially self-expandable. For example, the frame 504 may be shape-set in an expanded form such that the implant naturally expands following removal of external force from the delivery device. In some examples, the implant 500 may be balloon-expandable and/or may be configured to be expanded using any suitable devices and/or methods (e.g., expansion balloons and/or fluid-filled cuffs). For example, a balloon may be situated within the lumen of the implant 500 following removal of the implant from the delivery device and/or the balloon may be inflated to cause expansion of the implant 500.

The implant 500 may be configured to be retrievable following delivery. For example, the implant 500 may be configured to collapse into a delivery device (e.g., a catheter) following deployment in the IVC. In some examples, a therapeutic effect of the implant 500 may be evaluated by any suitable means following deployment of the implant 500. If the deployment is determined to be sub-optimal, the implant 500 may be retrieved and/or re-placed.

In some examples, the implant 500 may have any suitable width and/or diameter and/or may be configured to expand to any suitable width and/or diameter. The implant 500 may be configured to be sized for differently sized IVCs and/or for different patients.

The implant 500 and/or at least a portion of the implant 500 may be at least partially composed of and/or may be configured to be at least partially coated by one or more materials configured to inhibit thrombus formation. For example, the covering 502 may be at least partially composed of a material configured to inhibit thrombus formation and/or may be configured to be coated and/or may comprise a coating of a material configured to inhibit thrombus formation.

The implant 500 may have any suitable length between a first end 501 and a second end 503. In some examples, the length of the implant 500 may be determined to allow the implant 500 to extend across multiple hepatic veins.

In some examples, the implant 500 may be at least partially composed of one or more metals, which can include one or more shape-memory alloys (e.g., Nitinol). For example, the frame 504 of the implant 500 may be at least partially composed of Nitinol and/or stainless steel.

FIG. 6 illustrates an example flow-regulating implant 600 configured for placement at least partially within an IVC of a patient in accordance with one or more examples. The implant 600 may be configured to extend within and/or along at least a portion of an IVC and/or at an in-flow portion of the IVC. For example, the implant 600 may be configured to expand at least partially across one or more junction portions between one or more hepatic veins and the IVC.

The flow-regulating implant 600 may be configured for reducing hepatic vein blood flow into the IVC. The implant can have a tubular and/or cylindrical form to approximate a shape of the IVC and/or can provide radial blood flow regulation. For example, the implant 600 may be configured to regulate blood that is generally perpendicular and/or into the sides of the implant 600. The implant can comprise one or more openings 605 and/or apertures which can allow the implant 600 to partially block blood flow while allowing partial flow through the sides of the implant 600. The number and/or size of the openings 605 can vary along the implant 600 to allow for adjustment of flow rate based on placement of the implant 600 within the IVC.

The implant 600 may be configured to be positioned to cover one or more junctions between the IVC and at least one hepatic vein. In some examples, the implant 600 may be configured to at least partially block and/or allow blood flow from multiple hepatic veins.

In some examples, the implant 600 may comprise a wire frame 604 at least partially enclosed by a covering 602. The frame 604 may comprise a network of one or more interconnected wires forming a tubular and/or cylindrical shape and/or extending around a generally tubular and/or cylindrical lumen. The frame 604 may be composed of one or more materials, which can include one or more shape-memory metallic alloys (e.g., Nitinol). As shown in FIG. 6, the frame 604 and/or segments of the frame 604 may have a generally wavy and/or bent form and/or may be configured to compress and/or expand to facilitate delivery and/or placement of the implant 600 within the IVC. In some examples, the frame 604 may be shape-set in a tubular form and/or may be shaped into the tubular form by the covering 602 and/or otherwise. Segments of the frame 604 may be separated and/or gaps may exist between portions of the frame 604.

The covering 602 may be configured to extend over the frame 604 and/or to cover gaps between portions of the frame. In some examples, the covering 602 may have a tubular form and/or may be configured to adhere and/or otherwise attach to the frame 604 to assume the tubular form shown in FIG. 6. The covering 602 may be composed of any suitable material, including various fabrics. In some examples, the covering 602 may be at least partially fluid-tight and/or may be configured to prevent blood flow into the implant 600.

The openings 605 may represent punctures through the covering 602 and/or frame 604. For example, the covering 602 may be configured to be punctured without compromising and/or modifying the form and/or structure of the covering 602. In some examples, the covering 602 may comprise openings 605 at only a portion of the covering 602. For example, only portions of the covering 602 configured for placement over an in-flow tube from a hepatic vein may comprise openings 605. However, the covering 602 may comprise openings 605 at any and/or all portions of the covering 602. While the openings 605 are shown having a circular shape, the openings 605 may have any suitable shape and/or size.

In some examples, the implant 600 a number and/or size of openings 605 of the implant 600 may vary across a length of the implant. For example, the implant 600 can include two or more sections, with each section comprising a different number and/or size of openings 605. In the example shown in FIG. 6, the implant 600 may comprise three sections, including a first section 606, a second section 608, and/or a third section 610. However, the implant 600 may comprise any number of sections. In some examples, the implant 600 may comprise a single covering 602. Alternatively, the implant 600 may comprise multiple different coverings 602 and/or distinct sections of the covering 602. For example, each section of the first section 606, the second section 608, and/or the third section 610 may be distinct from each other.

As shown in FIG. 6, a number of openings 605 may vary across the different sections of the implant 600. For example, the first section 606 may comprise the most (e.g., approximately nine) openings 605. The second section 608 may comprise an intermediate number (e.g., six) of openings 605. The third section 610 may comprise the least (e.g., approximately four) openings 605.

The sections may be sized such that only one of the sections may be covering a single junction between the IVC and a hepatic vein. For example, the sections may be sized to be approximately as large or larger than a hepatic vein in-flow junction. In some examples, the implant 600 may be configured to be adjusted and/or moved following delivery into the IVC to modify an amount of blood flow obstruction caused by the implant 600. For example, the implant 600 may be initially deployed with the second section 608 covering a hepatic vein in-flow junction. Following deployment, an effect of the implant 600 on blood flow into the IVC may be evaluated. If the amount of blood flow obstruction caused by the implant 600 and/or an amount of blood volume in the splanchnic reservoir is less than desired, the implant 600 may be adjusted such that the first section 606, and not the second section 608, covers the hepatic vein in-flow junction. If the amount of blood flow obstruction caused by the implant 600 and/or an amount of blood volume in the splanchnic reservoir is more than desired, the implant 600 may be adjusted such that the third section 610, and not the second section 608 and/or first section 606, covers the hepatic vein in-flow junction. In examples in which the first section 606, second section 608, and/or third section 610 are at least partially aligned longitudinally (as shown in FIG. 6), the implant 600 may be configured to raised and/or lowered within an IVC to selectively position one of the sections across in-flow junctions of one or more hepatic veins.

While the sections of the implant 600 are shown extending along a length of the implant 600 and/or longitudinally at a first side of the implant 600, the implant 600 may comprise different sections spaced radially and/or axially around the implant 600. For example, the implant 600 may comprise a first section including a first number of openings 605 at a first side of the implant 600 and/or may comprise a second section including a different number of openings 605 from the first section at a second side of the implant (e.g., opposite the first side and/or in line with the first section). Accordingly, to adjust an amount of blood flow obstruction, the implant 600 may be configured to be twisted to modify which of the two sections (or more sections) is covering the hepatic vein in-flow junction.

In some examples, the implant 600 may be configured to at least partially obstruct all hepatic vein junctions with the IVC along the length of the implant 600. For example, the implant 600 may be configured to extend across IVC-hepatic vein junctions of two or three hepatic veins and/or may comprise one or more openings 605 at each junction. In some examples, the implant 600 may have a generally flexible and/or bendable structure and/or may otherwise be configured to facilitate delivery into the IVC.

While the covering 602 is shown comprising a single covering 602 with puncture openings through the covering 602, the covering 602 may additionally or alternatively comprise multiple disparate sections. For example, the covering 602 may comprise one or more strips and/or lengths of material with gaps between the strips and/or length of the material to allow blood flow through the gaps. In such examples, the covering 602 and/or coverings 602 may be configured to at least partially block in-flow from one or more hepatic veins and/or to not completely cover in-flow from the one or more hepatic veins. In some examples, the covering 602 may comprise one or more overlapping and/or intersecting strips and/or fibers of material forming gaps between the overlapping and/or intersection strips and/or fibers to allow some measure of blood flow through the covering 602. The covering 602 may comprise one or more cells configured to allow blood flow through the covering 602.

In some examples, the implant 600 may be configured to assume a compressed form while within a delivery device (e.g., a catheter). Following removal from the delivery device, the implant 600 may be configured to at least partially expand and/or otherwise anchor within the IVC. In some examples, the implant 600 may be at least partially self-expandable. For example, the frame 604 may be shape-set in an expanded form such that the implant naturally expands following removal of external force from the delivery device. In some examples, the implant 600 may be balloon-expandable and/or may be configured to be expanded using any suitable devices and/or methods. For example, a balloon and/or cuff may be situated within the lumen of the implant 600 following removal of the implant from the delivery device and/or the balloon and/or cuff may be inflated with gas and/or fluid to cause expansion of the implant 600.

The implant 600 may be configured to be retrievable following delivery. For example, the implant 600 may be configured to collapse into a delivery device (e.g., a catheter) following deployment in the IVC. In some examples, a therapeutic effect of the implant 600 may be evaluated by any suitable means following deployment of the implant 600. If the deployment is determined to be sub-optimal, the implant 600 may be retrieved and/or re-placed.

In some examples, the implant 600 may have any suitable width and/or diameter and/or may be configured to expand to any suitable width and/or diameter. The implant 600 may be configured to be sized for differently sized IVCs and/or for different patients.

The implant 600 and/or at least a portion of the implant 600 may be at least partially composed of and/or may be configured to be at least partially coated by one or more materials configured to inhibit thrombus formation. For example, the covering 602 may be at least partially composed of a material configured to inhibit thrombus formation and/or may be configured to be coated and/or may comprise a coating of a material configured to inhibit thrombus formation.

The implant 600 may have any suitable length between a first end 601 and a second end 603. In some examples, the length of the implant 600 may be determined to allow the implant 600 to extend across multiple hepatic veins. The implant 600 may be configured to allow blood flow longitudinally (e.g., from the first end 601 to the second end 603 and/or from the second end 603 to the first end 601) through the lumen of the implant 600 and/or laterally (e.g., through the openings 605) through the sides of the implant 600 and/or into the lumen of the implant 600.

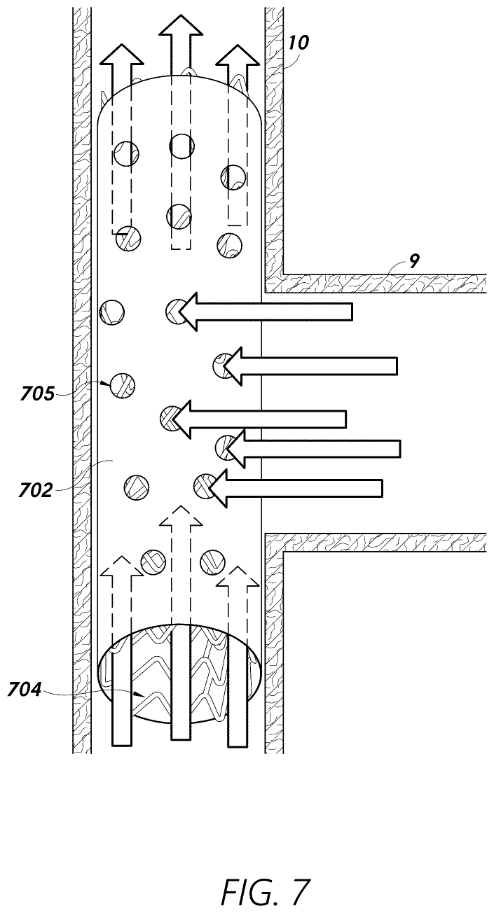

FIG. 7 illustrates an example flow-regulating implant deployed at least partially within an IVC 10 in accordance with one or more examples. The implant may comprise a frame 704 and/or a covering 702 at lease partially enclosing the frame 704. The frame 704 and/or covering 702 may have a generally tubular and/or cylindrical shape and/or may be at least partially compressible and/or expandable. In some examples, the frame 704 and/or covering 702 may comprise one or more puncture openings 705 configured to allow blood flow through the sides of the implant. The implant may be configured to allow longitudinal blood flow (e.g., towards the top of the page in FIG. 7) through the implant and/or lateral blood flow (e.g., towards the left side of the page in FIG. 7) through the sides of the implant (e.g., through the covering 702 and/or frame 704).

In some examples, the implant may be configured to extend at least partially across one or more junctions between the IVC 10 and one or more hepatic veins 9. For example, blood flow from one or more hepatic veins 9 may be prevented from entering the IVC 10 by the covering 702 and/or frame 704 of the implant. However, some limited amount of blood flow from the one or more hepatic veins 9 may be enabled to pass through openings 705 of the implant and/or to enter the IVC 10 and/or a lumen of the implant. Once inside the IVC 10 and/or the lumen of the implant, the blood may be carried upward along the IVC 10 with the natural upward blood flow of the IVC.

The position of the implant may be adjusted following deployment of the implant within the IVC 10. For example, the implant may be configured such that a length of the implant exceeds a width of a hepatic vein 9. Thus, the implant may be adjusted upward and/or downward within the IVC 10 while maintaining complete and/or partial coverage of the hepatic vein 9. By adjusting the position of the implant, a number and/or size of openings 705 available to the hepatic vein 9 may change, thus adjusting an amount of blood flow allowed by the implant from the hepatic vein 9 into the IVC 10 and/or lumen of the implant.

The implant may be configured for delivery via the IVC 10 and/or may be deployed at a position such that the implant simultaneously obstructs multiple hepatic veins 9. In some examples, the implant may not comprise any openings 705 and/or may allow blood flow through cells formed by one or more struts of the frame 704. For example, the implant may not comprise a covering and/or may be configured to allow partial blood flow through the frame 704 of the implant.

In some examples, the implant may be configured to be expanded using a balloon expander and/or other means. The implant may be configured to be retrievable following deployment within the IVC 10. In some examples, an impact on blood flow of the implant may be evaluated while the implant is tethered to a delivery system. If the blood flow impact is less than or more than desired, the implant may be retrieved and/or adjusted. Upon determination that a desired blood flow impact has been reached, any tethering between the delivery systems and the implant may be removed.

The implant may have any suitable size to allow the implant to fit within the IVC 10. In some cases, differently sized implants may be used depending on a determination of a size of a patient's IVC.

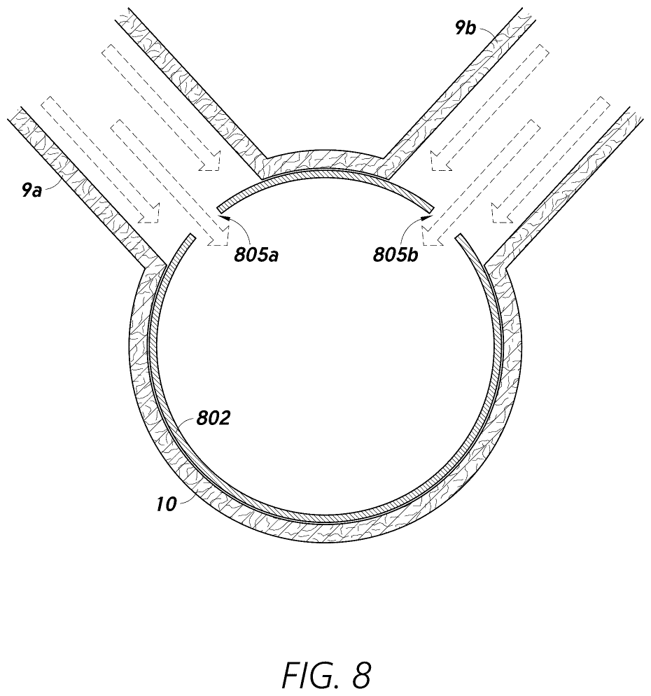

FIG. 8 illustrates an example flow-regulating implant deployed at least partially within an IVC 10 in accordance with one or more examples. The implant may comprise a frame and/or a covering 802. The frame and/or covering 802 may have a generally tubular and/or cylindrical shape and/or may be at least partially compressible and/or expandable. In some examples, the frame and/or covering 802 may comprise one or more puncture openings 805 configured to allow blood flow through the sides of the implant. The implant may be configured to allow blood flow longitudinal blood flow (e.g., into the page in FIG. 8) through the implant and/or lateral blood flow through the sides of the implant (e.g., through the covering 802 and/or frame).

In some examples, the implant may be configured to extend at least partially across one or more junctions between the IVC 10 and one or more hepatic veins 9. For example, the implant may be configured to extend at least partially and/or entirely across a first hepatic vein 9a and/or a second hepatic vein 9b. The first hepatic vein 9a and second hepatic vein 9b may be in-line across the implant and/or may be staggered along a length of the implant. Blood flow from the first hepatic vein 9a and/or second hepatic vein 9b may be prevented from entering the IVC 10 by the covering 802 and/or frame of the implant. However, some limited amount of blood flow from the first hepatic vein 9a and/or second hepatic vein 9b may be enabled to pass through openings 805 of the implant and/or to enter the IVC 10 and/or a lumen of the implant. Blood flow from the first hepatic vein 9a may pass through at least a first opening 805a of the implant and/or blood flow from the second hepatic vein 9b may pass through at least a second opening 805b of the implant. Once inside the IVC 10 and/or the lumen of the implant, the blood may be carried upward along the IVC 10 with the natural upward blood flow of the IVC.

The first opening 805a may be associated with a first portion of the covering 802 and/or the second opening 805b may be associated with a second portion of the covering 802. For example, the first portion may comprise a first amount, size, and/or shape of openings 805 and/or the second portion may comprise a second amount, size, and/or shape of openings 805 and/or the first amount, size, and/or shape of openings 805 may be greater or less than a second amount, size, and/or shape of openings 805. The first portion and the second portion may be at least partially aligned axially along the covering 802, as shown in FIG. 8. For example, by twisting the covering 802, the first opening 805a and/or first portion or the second opening 805b and/or second portion may selectively be positioned across the first hepatic vein 9a and/or second hepatic vein 9b.

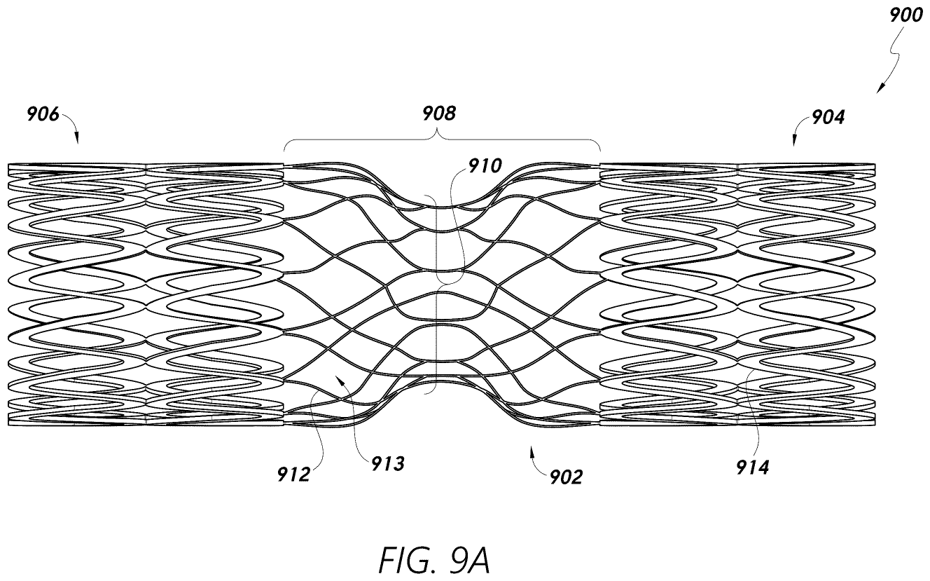

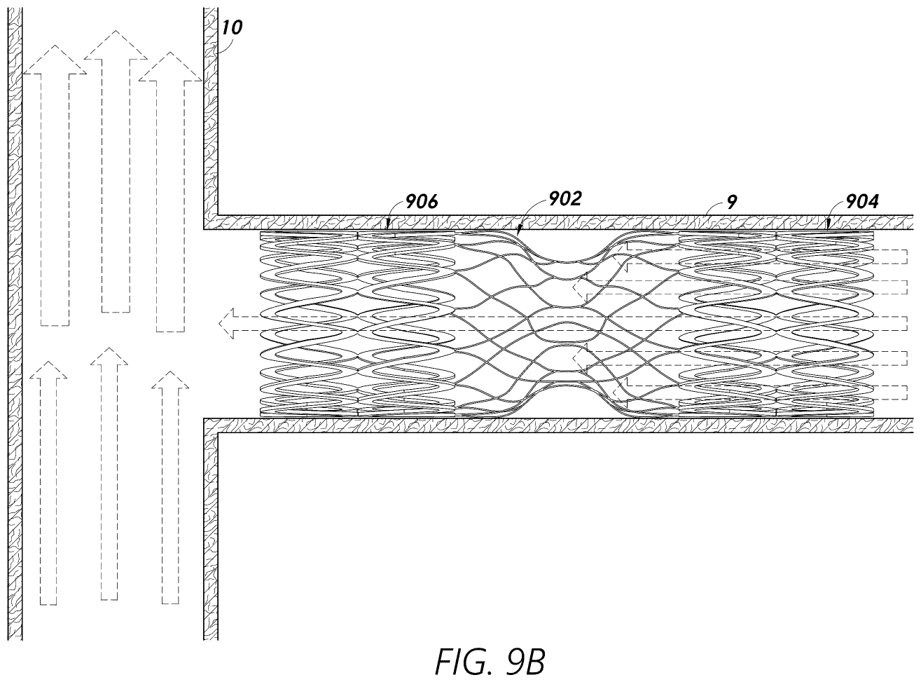

FIGS. 9A and 9B illustrate an example flow-regulating implant 900 configured to placement at least partially within a hepatic vein 9 to limit blood flow into an IVC 10 in accordance with one or more examples. In some examples, the implant 900 may be configured for use in conjunction with one or more implants situated at least partially within the IVC (e.g., the example implants described in FIGS. 5-8). However, the implant 900 may be configured for use independent of other implants. In some examples, the implant 900 may be configured to extend at least partially within the IVC 10.

The implant 900 may comprise one or more components, which can include a first (e.g., proximal) anchor 904, a second (e.g., distal) anchor 906, and/or a shunt portion 902 (e.g., midsection). The shunt portion 902 may be configured to be positioned at least partially between the first anchor 904 and the second anchor 906. In some examples, the first anchor 904, second anchor 906, and/or shunt portion 902 may be separate components which may be coupled together. However, the first anchor 904, second anchor 906, and/or shunt portion 902 may be extensions of a single device. For example, the shunt portion 902 may extend into the first anchor 904 and/or second anchor 906.

In some examples, the shunt portion 902 may have an hourglass shape. For example, the shunt portion 902 may have a variable width in which the shunt portion 902 may have a maximum width at one or more end portions of the shunt portion 902 and/or may reduce to a minimum width at a relatively narrow adjustable neck 910 and/or orifice. The neck 910 may be configured to limit and/or manage blood flow through the implant 900. The neck 910 may comprise an orifice configured to allow at least partial blood flow through the neck 910. In some examples, the neck 910 may represent and/or comprise a portion of the shunt portion 902 having reduced diameter and/or width with respect to other portions of the shunt portion 902. The shunt portion 902 may be configured to proportionately adjust an amount of blood flow through the shunt portion 902 based at least in part on a width and/or diameter of the shunt portion 902. For example, as the width of the shunt portion 902 decreases, the amount of blood flow through the shunt portion 902 may proportionately decrease. Moreover, as the width of the shunt portion 902 increases, the amount of blood flow through the shunt portion 902 may proportionately increase.

The shunt portion 902 can have an at least partially adjustable width and/or length. For example, a width and/or diameter of the neck 910 and/or a length 908 of the shunt portion 902 can be adjusted. In some examples, the width and/or diameter of the neck 910 and/or a length 908 of the shunt portion 902 can be adjusted based at least in part on a distance between the first anchor 904 and the second anchor 906. For example, as the first anchor 904 and the second anchor 906 separate, the length 908 of the shunt portion 902 can increase and/or a width and/or diameter of the neck 910 can increase as well. Moreover, as the first anchor 904 and the second anchor 906 move closer together, the length 908 of the shunt portion 902 can decrease and/or a width and/or diameter of the neck 910 can decrease as well.

In some examples, the shunt portion 902 can be composed of a network of struts 912, which can form one or more cells 913 between the struts 912. The struts 912 may comprise generally than wires and/or wire-like materials. In some examples, the struts 912 may be at least partially flexible and/or may be configured to bend in response to pressure from the first anchor 904, second anchor 906, the hepatic vein 9, and/or various delivery devices and/or anatomy features.

The shunt portion 902, first anchor 904, and/or second anchor 906 may be configured to be shape-set to a desired form. For example, the shunt portion 902 may be configured to be shape-set in the hourglass form shown in FIGS. 9A and 9B. In some examples, the implant 900 may be at least partially composed of one or more shape-memory alloys (e.g., Nitinol). During delivery, the implant 900 may be configured to assume a compressed form in which the implant 900 may have a reduced diameter and/or width. As the implant 900 is deployed within the hepatic vein 9, the implant 900 may be configured to naturally expand to the shape-set form and/or may press against the walls of the hepatic vein 9. In some examples, the implant 900 may be configured to expand via a balloon expander and/or other mechanical process.

The first anchor 904 and/or second anchor 906 may be at least partially composed of network of posts 914 and/or struts (wires and/or wire-like struts), which may be configured to bend to allow for compression and/or expansion of the first anchor 904 and/or second anchor 906. The posts 914 may be configured to form an accordion shape and/or may be shape-set in a circular and/or tubular form. The first anchor 904 and/or second anchor 906 may be configured to naturally expand upon removal from a delivery device (e.g., a catheter) and/or may expand to the diameter and/or width of the hepatic vein 9 such that the first anchor 904 and/or second anchor 906 may be configured to anchor to the walls of the hepatic vein 9 using frictional force and/or other means.

In some examples, the shunt portion 902 may comprise a covering at an inner side and/or outer side of the shunt portion 902. The covering may be configured to at least partially cover and/or close the cells 913 formed by the struts 912 of the shunt portion 902. As a result the shunt portion 902 may be configured to block blood flow through the cells 913 of the shunt portion 902 and/or only blood flow through the neck 910 of the shunt portion 902 may be allowed. As the neck 910 increases in width and/or diameter, blood flow through the implant 900 may increase. As the neck 910 decreases in width and/or diameter, blood flow through the implant 900 may decrease. In this way, the shunt portion 902 may be configured to limit and/or manage blood flow through the implant 900, through the hepatic vein 9, and/or into the IVC 10. In some examples, the shunt portion 902 can comprise a liner material composed at least partially of a polymer and/or metal such that the liner material (e.g., covering) may be impervious to blood flow. Additionally or alternatively, the struts 912 of the shunt portion 902 may be sufficiently compacted such the cells 913 allow minimal blood flow through the cells 913.

The implant 900 may be at least partially composed of one or more metals, which can include one or more shape-memory alloys (e.g., Nitinol) and/or stainless steel. For example the shunt portion 902, first anchor 904, and/or second anchor 906 may be at least partially composed of Nitinol and/or stainless steel.

In some examples, one or more diameters and/or widths of the implant 900 may be adjusted following deployment of the implant 900. For example, a balloon expander may be used to expand one or more portions (e.g., the neck 910) following deployment of the implant 900.

The shunt portion 902 may be configured to be expanded using one or more balloon expanders. For example, an hourglass-shaped balloon may be configured to fit within the shunt portion 902 and/or to approximate a shape of the shunt portion 902. Additionally or alternatively, a generally flat balloon expander may be used to increase a width and/or diameter of at least the neck 910 of the shunt portion 902.

The first anchor 904 and/or second anchor 906 may be configured to convey a relatively high radial force on the shunt portion 902 to hold the shunt portion 902 in a desired configuration and/or shape.

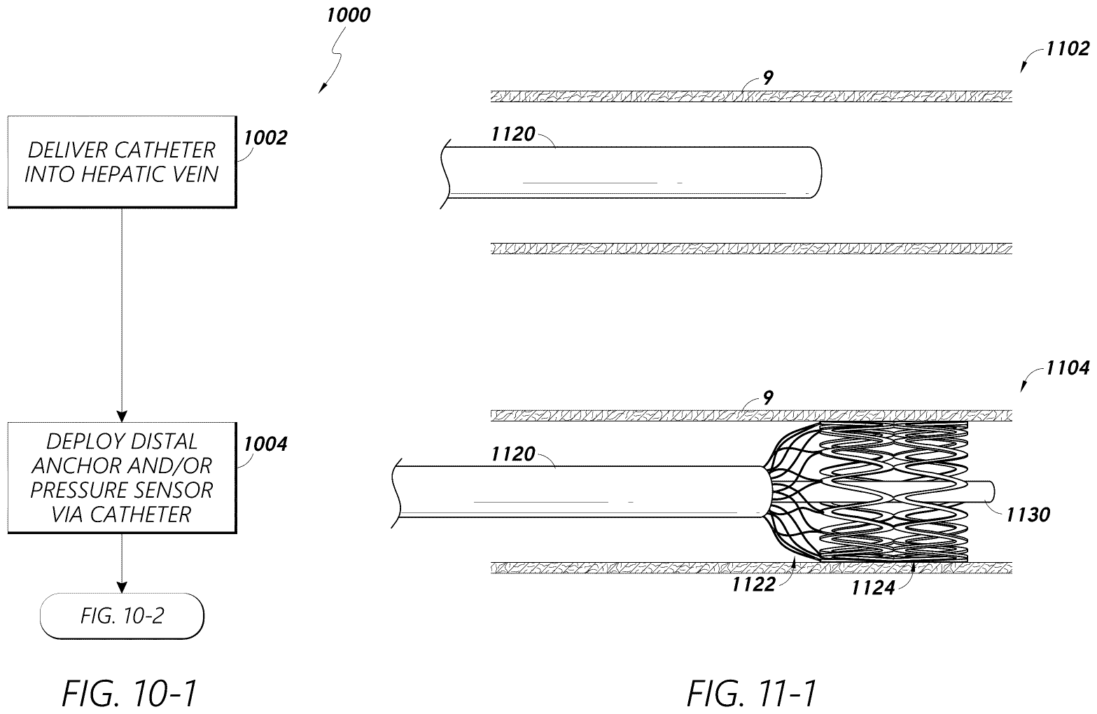

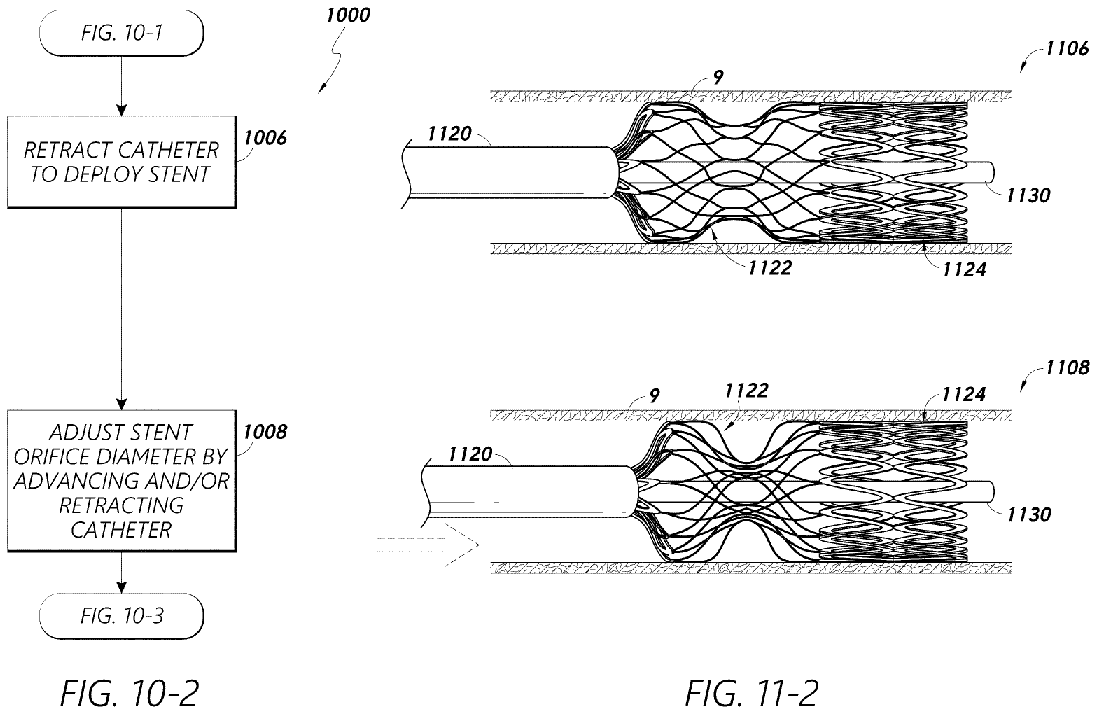

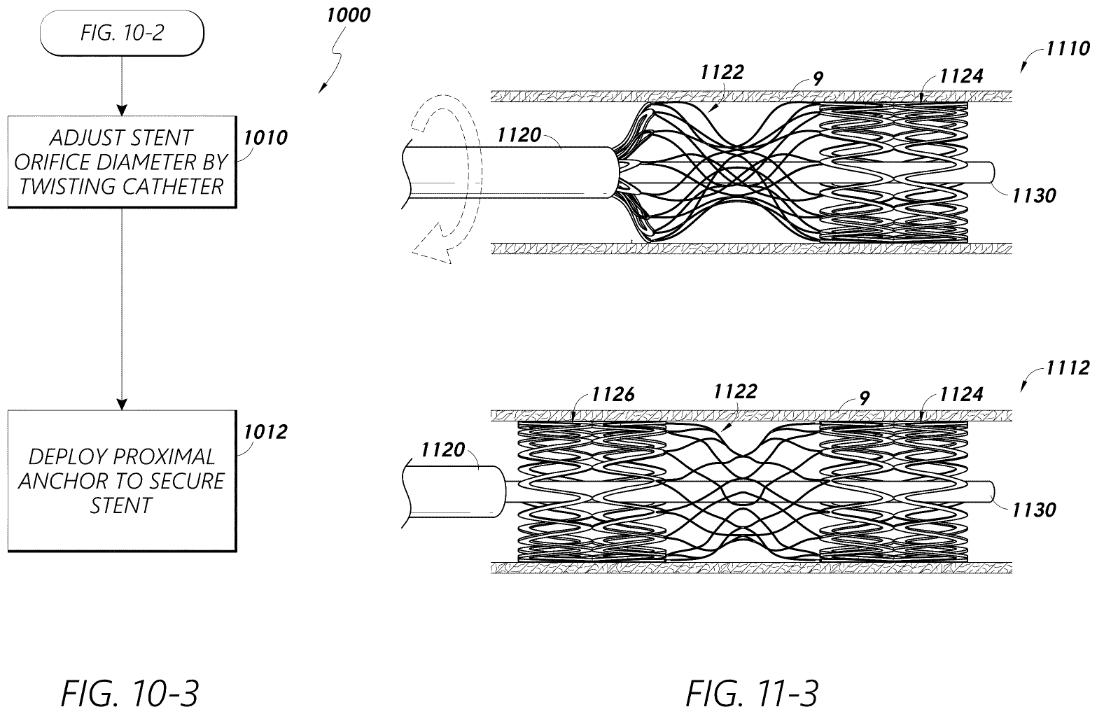

FIG. 10 (FIGS. 10-1, 10-2, and 10-3) provide a flowchart including steps of a process 1000 for delivering one or more implants in accordance with one or more examples. FIG. 11 (FIGS. 11-1, 11-2, and 11-3) provide images relating to various steps of the process 1000 of FIG. 10.

At step 1002, the process 1000 involves delivering a catheter 1120 into a hepatic vein 9, as illustrated in image 1102 of FIG. 11. The catheter 1120 may be delivered via an IVC into the hepatic vein 9 and/or may be delivered via any suitable means. The catheter 1120 may be configured to carry and/or deploy one or more implants for use in managing blood flow within and/or out of the hepatic vein 9. In some examples, the catheter 1120 may be a balloon catheter configured to at least partially expand a shunt portion 1122 and/or other portions of the implant.

At step 1004, the process 1000 involves deploying a first (e.g., distal) anchor 1124 out of the catheter 1120 and/or into the hepatic vein 9, as shown in image 1104 of FIG. 11. In some examples, delivery of the first anchor 1124 and/or additional implants and/or components via the catheter 1120 may be facilitated through use of a pressure sensor and/or other devices. In some examples, the first anchor 1124 and/or other components of an implant may be crimped onto and/or around a shaft 1130 configured to extend through and/or beyond the catheter 1120. One or more pressure sensors may be configured to couple to a distal end of the shaft 1130 and/or beyond the first anchor 1124. In some examples, a first sensor may be deployed distally of the first anchor 1124 and/or a second sensor may be deployed proximally of a second anchor 1126 to determine a pressure difference and/or to evaluate a change in blood flow caused by the implant.

The first anchor 1124 and/or other components of an implant may be configured to assume a compressed form while within the catheter 1120. Upon removal from the catheter 1120, the first anchor 1124 may be configured to expand and/or be expanded until the first anchor 1124 contacts and/or anchors to the walls of the hepatic vein 9. The first anchor 1124 may have a generally circular form and/or may approximate a shape of the hepatic vein 9.

At step 1006, the process 1000 involves at least partially retracting the catheter 1120 and/or otherwise deploying a shunt portion 1122 of the implant beyond a distal end of the catheter 1120, as shown in image 1106 of FIG. 11. The shunt portion 1122 may be configured to at least partially expand upon removal from the catheter 1120. In some examples, the shunt portion 1122 may be configured to expand until the shunt portion 1122 contacts the walls of the hepatic vein 9. However, the shunt portion 1122 may not necessarily contact the walls of the hepatic vein 9 and/or may be configured to anchor to the hepatic vein 9 via the first anchor 1124 and/or a second anchor 1126 of the implant.

At step 1008, the process 1000 involves (e.g., prior to deploying a second anchor 1126 from the catheter 1120) adjusting a diameter and/or width of a neck of the shunt portion 1122 and/or adjusting a length of the shunt portion 1122 by advancing and/or retracting the catheter 1120, as shown in image 1108 of FIG. 11. For example, by advancing the catheter 1120, a distance between the first anchor 1124 and a second anchor 1126 of the implant may decrease and/or the catheter 1120 may press against the shunt portion 1122 to reduce a length and/or width of the shunt portion 1122. Similarly, by retracting the catheter 1120, a distance between the first anchor 1124 and a second anchor 1126 of the implant may increase and/or a length and/or width of the shunt portion 1122 may be increased. The catheter 1120 may be adjusted until a desired amount of flow through the implant is reached.

At step 1010, the process 1000 involves (e.g., prior to deploying a second anchor 1126 from the catheter 1120) twisting the catheter 1120 to adjust the orifice diameter and/or width of the neck of the shunt portion 1122, as shown in image 1110 of FIG. 11. In some examples, twisting of the catheter 1120 may be performed in addition to and/or in place of advancement and/or retraction of the catheter 1120. By twisting the catheter 1120, the shunt portion 1122 may correspondingly be twisted and/or struts of the shunt portion 1122 may compress to reduce the width and/or diameter of the shunt portion 1122 orifice and/or the struts may expand to increase the width and/or diameter of the shunt portion 1122 orifice. Twisting the catheter 1120 and/or shunt portion 1122 in a first direction may cause reduction of the length and/or width of the shunt portion 1122 and/or twisting the catheter 1120 and/or shunt portion 1122 in a second direction may cause an increase of the length and/or width of the shunt portion 1122. The catheter 1120 may be twisted in either direction until a desired amount of flow through the implant is reached.

At step 1012, the process 1000 involves deploying a second (e.g., proximal) anchor 1126 beyond a distal end of the catheter 1120, as shown in image 1112 of FIG. 11. The second anchor 1126 may be configured to expand and/or otherwise anchor to the walls of the hepatic vein 9 to secure the shunt portion 1122 in place.

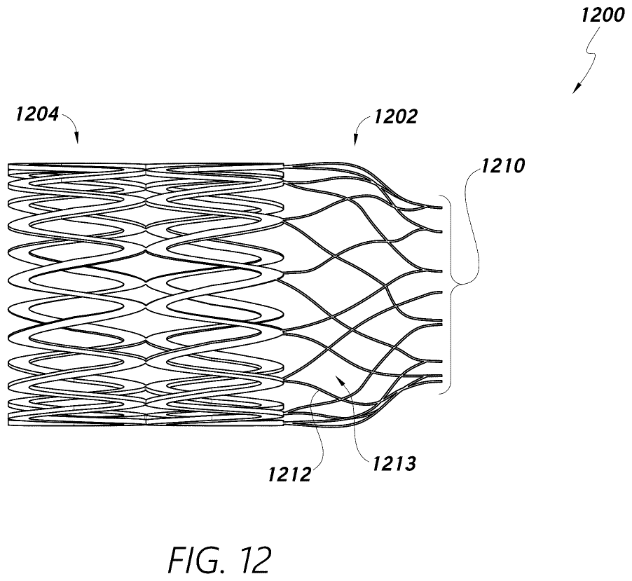

FIG. 12 illustrates another example flow-regulating implant 1200 configured to placement at least partially within a hepatic vein and/or other blood vessel to limit blood flow into an IVC 10 and/or other blood vessel in accordance with one or more examples. In some examples, the implant 1200 may be configured for use in conjunction with one or more implants situated at least partially within the IVC (e.g., the example implants described in FIGS. 5-8). However, the implant 1200 may be configured for use independent of other implants. In some examples, the implant 1200 may be configured to extend at least partially within the IVC 10.

The implant 1200 may comprise one or more components, which can include an anchor 1204 and/or a shunt portion 1202. In some examples, the anchor 1204 and/or shunt portion 1202 may be separate components which may be coupled together. However, the anchor 1204 and/or shunt portion 1202 may be extensions of a single device. For example, the shunt portion 1202 may extend into the anchor 1204. The anchor 1204 may be configured for placement upstream or downstream of the shunt portion 1202.

In some examples, the shunt portion 1202 may have a partial hourglass shape. For example, the shunt portion 1202 may have a variable width in which the shunt portion 1202 may have a maximum width at one or more end portions of the shunt portion 1202 (e.g., where the shunt portion 1202 meets the anchor 1204) and/or may reduce to a minimum width at a relatively narrow neck 1210. The neck 1210 may be configured to limit and/or manage blood flow through the implant 1200.

The shunt portion 1202 can have an at least partially adjustable width and/or length. For example, a width and/or diameter of the neck 1210 and/or a length of the shunt portion 1202 can be adjusted.

In some examples, the shunt portion 1202 can be composed of a network of struts 1212, which can form one or more cells 1213 between the struts 1212. The struts 1212 may comprise generally than wires and/or wire-like materials. In some examples, the struts 1212 may be at least partially flexible and/or may be configured to bend in response to pressure from the anchor 1204, the hepatic vein, and/or various delivery devices and/or anatomy features.

The shunt portion 1202 and/or anchor 1204 may be configured to be shape-set to a desired form. For example, the shunt portion 1202 may be configured to be shape-set in the partial hourglass form shown in FIG. 12. In some examples, the implant 1200 may be at least partially composed of one or more shape-memory alloys (e.g., Nitinol). During delivery, the implant 1200 may be configured to assume a compressed form in which the implant 1200 may have a reduced diameter and/or width. As the implant 1200 is deployed within a blood vessel, the implant 1200 may be configured to naturally expand to the shape-set form and/or may press against the walls of the blood vessel. In some examples, the implant 1200 may be configured to expand via a balloon expander and/or other mechanical process.

The anchor 1204 may be at least partially composed of a network of posts, which may be configured to bend to allow for compression and/or expansion of the anchor 1204. The posts may be configured to form an accordion shape and/or may be shape-set in a circular and/or tubular form. The anchor 1204 may be configured to naturally expand upon removal from a delivery device (e.g., a catheter) and/or may expand to the diameter and/or width of the blood vessel such that the anchor 1204 may be configured to anchor to the walls of the blood vessel using frictional force and/or other means.

In some examples, the shunt portion 1202 may comprise a covering at an inner side and/or outer side of the shunt portion 1202. The covering may be configured to at least partially cover and/or close the cells 1213 formed by the struts 1212 of the shunt portion 1202. As a result the shunt portion 1202 may be configured to block blood flow through the cells 1213 of the shunt portion 1202 and/or only allow blood flow through the neck 1210 of the shunt portion 1202. As the neck 1210 increases in width and/or diameter, blood flow through the implant 1200 may increase. As the neck 1210 decreases in width and/or diameter, blood flow through the implant 1200 may decrease. In this way, the shunt portion 1202 may be configured to limit and/or manage blood flow through the implant 1200, through the blood vessel, and/or into a branching blood vessel. In some examples, the shunt portion 1202 can comprise a liner material composed at least partially of a polymer and/or metal such that the liner material (e.g., covering) may be impervious to blood flow. Additionally or alternatively, the struts 1212 of the shunt portion 1202 may be sufficiently compacted such the cells 1213 allow minimal blood flow through the cells 1213.

The implant 1200 may be at least partially composed of one or more metals, which can include one or more shape-memory alloys (e.g., Nitinol) and/or stainless steel. For example, the shunt portion 1202 and/or anchor 1204 may be at least partially composed of Nitinol and/or stainless steel.

In some examples, one or more diameters and/or widths of the implant 1200 may be adjusted following deployment of the implant 1200. For example, a balloon expander may be used to expand one or more portions (e.g., the neck 1210) following deployment of the implant 1200.

The shunt portion 1202 may be configured to be expanded using one or more balloon expanders. For example, a partial hourglass-shaped balloon may be configured to fit within the shunt portion 1202 and/or to approximate a shape of the shunt portion 1202. Additionally or alternatively, a generally flat balloon expander may be used to increase a width and/or diameter of at least the neck 1210 of the shunt portion 1202.

Some implementations of the present disclosure relate to a medical implant for managing blood flow. The medical implant comprises a frame configured for deployment within a first blood vessel and configured to extend at least partially across a first in-flow junction of a second blood vessel, the frame at least partially composed of wire-like struts configured to allow at least partial blood flow through gaps between struts.

In some examples, the medical implant further comprises a covering configured to at least partially enclose the frame. The covering may be configured to at least partially impede blood flow from the second blood vessel into the first blood vessel.

The covering may comprise one or more openings configured to allow blood flow through the covering.

In some examples, a first portion of the covering comprises a first amount of openings and a second portion of the covering comprises a second amount of openings. The first amount may be greater than the second amount.

A third portion of the covering may comprise a third amount of openings. The third amount may be less than the second amount.

In some examples, the frame is configured to be adjusted within the first blood vessel to selectively position one of the first portion and the second portion across the first in-flow junction of the second blood vessel.

The first portion and the second portion may be aligned axially along the covering. In some examples, the first portion and the second portion are aligned longitudinally along the covering.

In some examples, the frame is configured to extend at least partially across a second in-flow junction of a third blood vessel.

The frame may have a generally tubular shape to approximate a shape of the first blood vessel. In some examples, the frame comprises an inner lumen configured to allow blood within the first blood vessel through the frame.

In accordance with some implementations of the present disclosure, a medical implant for managing blood flow comprises a shunt portion configured for deployment within a first blood vessel. The shunt portion has an adjustable orifice and is configured to adjust blood flow through the first blood vessel based at least in part on a size of the adjustable orifice. The medical implant further comprises a first anchor coupled to the shunt portion within the first blood vessel and configured to anchor the shunt portion in place.

The first anchor may be configured for placement upstream of the shunt portion. The medical implant may further comprise a second anchor configured for placement downstream of the shunt portion within the first blood vessel and configured to anchor the shunt portion in place.

In some examples, a distance between the first anchor and the second anchor is adjustable. Advancing or retracting the second anchor with respect to the first anchor may modify a width of the adjustable orifice.

A width of the adjustable orifice may be modified by twisting the shunt portion. The shunt portion may have an hourglass shape in which the adjustable orifice represents a portion of reduced diameter of the shunt portion.

In some examples, the shunt portion comprises a network of struts forming one or more cells. The shunt portion may comprise a covering at least partially enclosing the network of struts and one or more cells.

The first anchor may be configured to self-expand from a compressed form within a delivery device to an expanded form following removal from the delivery device. In some examples, the shunt portion is configured to be expanded using an expansion balloon.

In some examples, the shunt portion is configured to be expanded using a fluid-filled cuff.

Some implementations of the present disclosure relate to a method comprising percutaneously delivering, via a catheter, a first anchor into a first blood vessel near a junction between the first blood vessel and a second blood vessel and percutaneously delivering, via the catheter, a shunt portion coupled to the first anchor into the first blood vessel downstream of the first anchor. The shunt portion has an adjustable orifice configured to adjust blood flow through the first blood vessel based at least in part on a size of the adjustable orifice.

The method may further comprise percutaneously delivering, via the catheter, a second anchor coupled to the shunt portion into the first blood vessel downstream of the shunt portion.

In some examples, the method further comprises, prior to percutaneously delivering the second anchor, advancing or retracting the catheter to adjust a distance between the first anchor and the second anchor and to adjust the size of the adjustable orifice. The method may further comprise, prior to percutaneously delivering the second anchor, twisting the catheter to adjust the size of the adjustable orifice.

The first anchor may be configured to self-expand from a compressed form within the catheter to an expanded form following removal from the catheter. In some examples, the method further comprises expanding the shunt portion using an expansion balloon.

In some examples, the method further comprises expanding the shunt portion using a fluid-filled cuff.

### Additional Description of Examples

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1: A medical implant for managing blood flow, the medical implant comprising a frame configured for deployment within a first blood vessel and configured to extend at least partially across a first in-flow junction of a second blood vessel, the frame at least partially composed of wire-like struts configured to allow at least partial blood flow through gaps between struts.

Example 2: The medical implant of any example herein, in particular example 1, further comprising a covering configured to at least partially enclose the frame, the covering configured to at least partially impede blood flow from the second blood vessel into the first blood vessel.

Example 3: The medical implant of any example herein, in particular example 2, wherein the covering comprises one or more openings configured to allow blood flow through the covering.

Example 4: The medical implant of any example herein, in particular example 3, wherein a first portion of the covering comprises a first amount of openings and a second portion of the covering comprises a second amount of openings, and wherein the first amount is greater than the second amount.

Example 5: The medical implant of any example herein, in particular example 4, wherein a third portion of the covering comprises a third amount of openings, and wherein the third amount is less than the second amount.

Example 6: The medical implant of any example herein, in particular example 4, wherein the frame is configured to be adjusted within the first blood vessel to selectively position one of the first portion and the second portion across the first in-flow junction of the second blood vessel.

Example 7: The medical implant of any example herein, in particular example 4, wherein the first portion and the second portion are aligned axially along the covering.

Example 8: The medical implant of any example herein, in particular example 4, wherein the first portion and the second portion are aligned longitudinally along the covering.

Example 9: The medical implant of any example herein, in particular example 1, wherein the frame is configured to extend at least partially across a second in-flow junction of a third blood vessel.

Example 10: The medical implant of any example herein, in particular example 1, wherein the frame has a generally tubular shape to approximate a shape of the first blood vessel.

Example 11: The medical implant of any example herein, in particular example 1, wherein the frame comprises an inner lumen configured to allow blood flow within the first blood vessel through the frame.

Example 12: The medical implant of any example herein, in particular example 1, further comprising a first anchor coupled to the frame within the first blood vessel and configured to anchor the frame in place.

Example 13: The medical implant of any example herein, in particular example 12, wherein the first anchor is configured for placement upstream of the frame, the medical implant further comprising a second anchor configured for placement downstream of the frame within the first blood vessel and configured to anchor the frame in place.

Example 14: The medical implant of any example herein, in particular example 13, wherein a distance between the first anchor and the second anchor is adjustable and wherein advancing or retracting the second anchor with respect to the first anchor modifies a width of an orifice of the frame.

Example 15: A medical implant for managing blood flow, the medical implant comprising a shunt portion configured for deployment within a first blood vessel, the shunt portion having an adjustable orifice and configured to adjust blood flow through the first blood vessel based at least in part on a size of the adjustable orifice and a first anchor coupled to the shunt portion within the first blood vessel and configured to anchor the shunt portion in place.

Example 16: The medical implant of any example herein, in particular example 15, wherein the first anchor is configured for placement upstream of the shunt portion, the medical implant further comprising a second anchor configured for placement downstream of the shunt portion within the first blood vessel and configured to anchor the shunt portion in place.

Example 17: The medical implant of any example herein, in particular example 16, wherein a distance between the first anchor and the second anchor is adjustable and wherein advancing or retracting the second anchor with respect to the first anchor modifies a width of the adjustable orifice.

Example 18: The medical implant of any of any example herein, in particular example 15, wherein a width of the adjustable orifice is modified by twisting the shunt portion.

Example 19: The medical implant of any example herein, in particular example 15, wherein the shunt portion has an hourglass shape in which the adjustable orifice represents a portion of reduced diameter of the shunt portion.

Example 20: The medical implant of any example herein, in particular example 15, wherein the shunt portion comprises a network of struts forming one or more cells.

Example 21: The medical implant of any example herein, in particular example 20, wherein the shunt portion comprises a covering at least partially enclosing the network of struts and one or more cells.

Example 22: The medical implant of any example herein, in particular example 15, wherein the first anchor is configured to self-expand from a compressed form within a delivery device to an expanded form following removal from the delivery device.

Example 23: The medical implant of any example herein, in particular example 15, wherein the shunt portion is configured to be expanded using an expansion balloon.

Example 24: The medical implant of any example herein, in particular example 15, wherein the shunt portion is configured to be expanded using a fluid-filled cuff.

Example 25: A method comprising percutaneously delivering, via a catheter, a first anchor into a first blood vessel near a junction between the first blood vessel and a second blood vessel and percutaneously delivering, via the catheter, a shunt portion coupled to the first anchor into the first blood vessel downstream of the first anchor, the shunt portion having an adjustable orifice configured to adjust blood flow through the first blood vessel based at least in part on a size of the adjustable orifice.

Example 26: The method of any example herein, in particular example 25, further comprising percutaneously delivering, via the catheter, a second anchor coupled to the shunt portion into the first blood vessel downstream of the shunt portion.

Example 27: The method of any example herein, in particular example 26, further comprising, prior to percutaneously delivering the second anchor, advancing or retracting the catheter to adjust a distance between the first anchor and the second anchor and to adjust the size of the adjustable orifice.

Example 28: The method of any example herein, in particular example 26, further comprising, prior to deploying the second anchor from the catheter, twisting the catheter to adjust the size of the adjustable orifice.

Example 29: The method of any example herein, in particular example 25, wherein the first anchor is configured to self-expand from a compressed form within the catheter to an expanded form following removal from the catheter.

Example 30: The method of any example herein, in particular example 25, further comprising expanding the shunt portion using an expansion balloon.

Example 31: The method of any example herein, in particular example 25, further comprising expanding the shunt portion using a fluid-filled cuff.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

Delivery systems as described herein may be used to position catheter tips and/or catheters to various areas of a human heart. For example, a catheter tip and/or catheter may be configured to pass from the right atrium into the coronary sinus. However, it will be understood that the description can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, such as for positioning within an atrium or coronary sinus, including for example delivery catheters and/or cannulas. It will be understood that other types of medical implant devices and/or procedures can be delivered to the coronary sinus using a delivery system as described herein, including for example ablation procedures, drug delivery and/or placement of coronary sinus leads.

What is claimed is:

1. A medical implant for managing blood flow, the medical implant comprising:
a frame configured for deployment within a first blood vessel and configured to extend at least partially across a first in-flow junction of a second blood vessel, the frame at least partially composed of wire-like struts configured to allow at least partial blood flow through gaps between struts.

2. The medical implant of claim 1, further comprising a covering configured to at least partially enclose the frame, the covering configured to at least partially impede blood flow from the second blood vessel into the first blood vessel.

3. The medical implant of claim 2, wherein the covering comprises one or more openings configured to allow blood flow through the covering, and wherein a first portion of the covering comprises a first amount of openings and a second portion of the covering comprises a second amount of openings, and wherein the first amount is greater than the second amount.

4. The medical implant of claim 1, wherein the frame is configured to extend at least partially across a second in-flow junction of a third blood vessel.

5. The medical implant of claim 1, wherein the frame has a generally tubular shape to approximate a shape of the first blood vessel.

6. The medical implant of claim 1, wherein the frame comprises an inner lumen configured to allow blood flow within the first blood vessel through the frame.

7. The medical implant of claim 1, further comprising a first anchor coupled to the frame within the first blood vessel and configured to anchor the frame in place, wherein the first anchor is configured for placement upstream of the frame, the medical implant further comprising a second anchor configured for placement downstream of the frame within the first blood vessel and configured to anchor the frame in place.

8. A medical implant for managing blood flow, the medical implant comprising:
a shunt portion configured for deployment within a first blood vessel, the shunt portion having an adjustable orifice and configured to adjust blood flow through the first blood vessel based at least in part on a size of the adjustable orifice; and
a first anchor coupled to the shunt portion within the first blood vessel and configured to anchor the shunt portion in place.

9. The medical implant of claim 8, wherein the first anchor is configured for placement upstream of the shunt portion, the medical implant further comprising a second anchor configured for placement downstream of the shunt portion within the first blood vessel and configured to anchor the shunt portion in place.

10. The medical implant of claim 8, wherein a width of the adjustable orifice is modified by twisting the shunt portion.

11. The medical implant of claim 8, wherein the shunt portion has an hourglass shape in which the adjustable orifice represents a portion of reduced diameter of the shunt portion.

12. The medical implant of claim 8, wherein the shunt portion comprises a network of struts forming one or more cells.

13. The medical implant of claim 8, wherein the first anchor is configured to self-expand from a compressed form within a delivery device to an expanded form following removal from the delivery device.

14. A method comprising:
percutaneously delivering, via a catheter, a first anchor into a first blood vessel near a junction between the first blood vessel and a second blood vessel; and
percutaneously delivering, via the catheter, a shunt portion coupled to the first anchor into the first blood vessel downstream of the first anchor, the shunt portion having an adjustable orifice configured to adjust blood flow through the first blood vessel based at least in part on a size of the adjustable orifice.

15. The method of claim 14, further comprising percutaneously delivering, via the catheter, a second anchor coupled to the shunt portion into the first blood vessel downstream of the shunt portion.

16. The method of claim 15, further comprising, prior to percutaneously delivering the second anchor, advancing or retracting the catheter to adjust a distance between the first anchor and the second anchor and to adjust the size of the adjustable orifice.

17. The method of claim 15, further comprising, prior to deploying the second anchor from the catheter, twisting the catheter to adjust the size of the adjustable orifice.

18. The method of claim 14, wherein the first anchor is configured to self-expand from a compressed form within the catheter to an expanded form following removal from the catheter.

19. The method of claim 14, further comprising expanding the shunt portion using an expansion balloon.

20. The method of claim 14, further comprising expanding the shunt portion using a fluid-filled cuff.
